(19) United States
(12) Patent Application Publication
(10) Pub. No.: US 2014/0079634 A1
(43) Pub. Date: Mar. 20, 2014

(54) LABELED PE2I FORMULATION AND METHOD

(71) Applicant: NAVIDEA BIOPHARMACEUTICALS INC., Dublin, OH (US)

(72) Inventors: Stephen B. Haber, Westford, MA (US); Richard Thorn, Mendon, MA (US)

(73) Assignee: NAVIDEA BIOPHARMACEUTICALS INC., Dublin, OH (US)

(21) Appl. No.: 14/026,366

(22) Filed: Sep. 13, 2013

Related U.S. Application Data
(60) Provisional application No. 61/702,294, filed on Sep. 18, 2012.

(51) Int. Cl. A61K 51/04; C07D 451/02

(52) U.S. Cl. CPC A61K 51/0455; C07D 451/02
USPC 424/1.85; 546/132

(57) ABSTRACT

A diagnostic formulation is provided comprising a tropane having a radioactive concentration of at least 1.6 mCi/mL at least about 51 hours post creation. The diagnostic formulation optionally comprises a radiolabeled dopamine transporter (DAT) ligand useful in the diagnosis of Parkinson's disease (PS). One example of a radiolabeled dopamine transporter (DAT) ligand example is [¹²³I]-N-(3-iodoprop-2E-enyl)-2β-carbomethoxy-3β-(4-methylphenyl) nortropane.

### FIELD OF THE INVENTION

This disclosure is in the field of medicine and in particular diagnostics of neurological disorders. This disclosure includes one or more formulations comprising an aqueous solution of N-(3-iodoprop-2E-enyl)-2β-carbomethoxy-3β-(4-methylphenyl)-nortropane ([¹²³I]-PE2I). Particular reference is made to such solution comprising a radioactive concentration of at least about 18 mCi/mL, and particularly about 20 mCi/mL or more.

### BACKGROUND OF THE INVENTION

N-(3-iodoprop-2E-enyl)-2b-carbomethoxy-3b-(4-methylphenyl)-nortropane ([¹²³I]-PE2I) is useful in diagnosing disorders involving changes in dopaminergic cells in the brain leading to alterations in levels of expression of the dopamine transporter (DAT), including Parkinson's Syndromes (PS), Dementia with Lewy Bodies (DLB) and Attention Deficit Hyperactivity Disorder (ADHD). Without being bound by any particular theory, these disorders are believed to be characterized by the loss of dopamine-producing neurons in the brain. The loss of dopamine-producing neurons is believed to begin long before symptoms of these diseases actually present. Symptoms of these diseases are often similar to other disorders. Consequently, misdiagnosis and non-diagnosis rates are high. Some sources report up to 50% misdiagnosis in the early stages. There is currently no available test that can clearly identify PS, DLB or ADHD, especially in early cases. A diagnostic for early stages of these disorders has long been sought.

Without being bound by any particular theory, the dopamine transporter (DAT) is believed to play a significant role in physiological, pharmacological and pathological processes in the brain. The transport system is a primary mechanism for terminating the effects of synaptic dopamine, thereby contributing to the maintenance of homeostasis in dopamine systems. It has also been reported to be a principal target of cocaine in the brain. (Kennedy and Hanbauer, J. Neurochem. 1983, 41, 172 178; Shoemaker et al., Naunyn-Schmeideberg's Arch. Pharmacol. 1985, 329, 227 235; Reith et al., Biochem Pharmacol. 1986, 35, 1123 1129; Ritz et al., Science 1987, 237, 1219 1223; Madras et al., J. Pharmacol. Exp. Ther. 1989a, 251, 131 141; Bergman et al., J. Pharmacol. Exp. Ther. 1989, 251, 150 155; Madras and Kaufman, Synapse 1994, 18, 261 275).

The brain grouping formed by the caudate nucleus and the putamen is called the striatum. It constitutes the major target for the cortical afferents of the basal ganglia. The striatum reportedly has the highest levels of dopamine terminals in the brain. A high density of DAT is localized on dopamine neurons in the striatum. DAT density has been reported to be a marker for a number of physiological and pathological states. For example, in Parkinson's Syndromes, dopamine is reportedly severely reduced and the depletion of DAT in the striatum has been an indicator for Parkinson's disease (Schoemaker et al., Naunyn-Schmeideberg's Arch. Pharmacol. 1985, 329, 227-235; Kaufman and Madras, Synapse 1991, 9, 43-49). Early or pre-symptomatic diagnosis of Parkinson's Syndromes has been reported employing the quantitative measurement of DAT depletion in the striatum. (Kaufman and Madras, Synapse 1991, 9, 43-49). DAT depletion has been reported measured by a noninvasive means such as brain imaging using a scintillation camera system and a suitable imaging agent (Frost et al., Ann. Neurology 1993, 34, 423 431; Hantraye et al., Neuroreport 1992, 3, 265-268; Costa, et al., “Dementia with Lewy bodies versus Alzheimer's disease: Role of dopamine transporter imaging,” Movement Disorders, 18(S7), S34-S38 (2004); and Walker et al., “Dementia with lewy bodies: A comparison of clinical diagnosis, FP-CIT SPECT imaging and autopsy” J Neurol Neurosurg Psychiatry 17353255 (Mar. 12, 2007).

The decay of the [¹²³I] associated with the compounds of this disclosure releases a photon reported to have an energy of 159 KeV. Such photons are capable of passing through human tissue. The photon emission is detectable with devices such as a radiation detector array in a Single Photon Emission Computed Tomography (SPECT) camera. With appropriate software, an image of the site from which the radiation is emerging is constructed. The image is usefully compared with other images including images from subjects without signs of the disorder. A decrease in emission is presumptive evidence of a loss of dopamine transporter neurons, and potentially indicative of PS, DLB and/or ADHD.

A useful imaging agent for the disorders described above exhibits a specific binding affinity and selectivity for the transporter being targeted. In addition, for imaging agents based on radioactive emission, a minimum level of radioactivity is also pertinent. The level of radioactivity is expressed in three ways: specific activity, the concentration of radioactivity, and the total amount of radioactivity administered. In addition, for a viable commercial product, it is advantageous for the radiochemical yield to be reasonable.

Specific activity, in this context, refers to the proportion of N-(3-iodoprop-2E-enyl)-2β-carbomethoxy-3β-(4-methylphenyl)nortropane molecules that have ¹²³I as opposed to ¹²⁷I, the non-radioactive iodine isotope. To maximize the amount of signal per bound molecule, it is advantageous in a radiochemical to minimize or exclude non-radioactive sodium iodide.

The concentration of the radiochemical and its stability are significant factors in the commercial viability of radio-chemicals. The radiochemical and chemical stability of each uniquely structured radio-labeled entity is unpredictable from structure alone. Furthermore, the effect of additives intended to stabilize the radiochemical solution cannot be known in advance of experimental testing.

Imaging agents that target the dopamine transporter include [¹²³I]-2β-carbomethoxy-3β-(4 fluorophenyl)-N-(3-iodo-E-allyl)nortropane ([¹²³I]-E-IACFT (CFT)), [¹²³I] N-ω-fluoropropyl-2β-carbomethoxy-3β-(4-iodophenyl)nortropane or Ioflupane (¹²³I) and [¹²³I]-N-(3-iodoprop-2E-enyl)-2β-carbomethoxy-3β-(4-methylphenyl) nortropane. Terminal sterilization via autoclaving is the preferred method of producing sterile formulations for injection. Some pharmaceutical formulations are, however, unstable under such conditions. Indeed, autoclaving Ioflupane (¹²³I) at 121° C. for 15 minutes led to a 20% decrease in radiochemical purity.

### SUMMARY OF THE INVENTION

In one aspect, the invention features a diagnostic formulation comprising an aqueous solution comprising [¹²³I]-N-(3-iodoprop-2E-enyl)-2β-carbomethoxy-3β-(4-methylphenyl)nortropane.

In one embodiment the formulation comprises a radioactive concentration of at least about 18-20 mCi/mL ab initio. In another embodiment, the formulation exhibits radioactive concentration of at least about 1.6 mCi/mL at least about 51 hours post-creation. In yet another embodiment the formulation comprises a pH of less than about 7. In another embodiment the formulation comprises a radiochemical purity of at least about 95%.

In another embodiment the formulation comprises a concentration of ethanol in a percentage of less than about 10%. In another embodiment the formulation is substantially carrier free. In another embodiment the formulation is substantially ascorbic acid free.

In a specific aspect, the invention features a method of preparing [¹²³I]-N-(3-iodoprop-2E-enyl)-2β-carbomethoxy-3β-(4-methylphenyl)nortropane comprising the steps of: a) Preparing a precursor solution comprising N-(3-tributyltin-2E-propenyl)-2β-carbomethoxy-3β-(4-methylphenyl)nortropane, ethanol, hydrogen peroxide, and phosphate buffer; b) Preparing a sodium [¹²³I]-iodide solution comprising sodium [¹²³I]-iodide and trifluoroacetic acid having a pH of less than about 2; and c) Heating a mixture of precursor solution and sodium [¹²³I]-iodide solution at a temperature of about 80° C. for about 15 minutes.

In another aspect, the invention features a method of preparing an aqueous solution of [¹²³I]-N-(3-iodoprop-2E-enyl)-2β-carbomethoxy-3β-(4-methylphenyl)nortropane comprising the steps of: eluting the [¹²³I]-N-(3-iodoprop-2E-enyl)-2β-carbomethoxy-3β-(4-methylphenyl)nortropane through a C18 preparative HPLC column with an eluent, wherein the eluent comprises about 15% (v/v) ethanol; and collecting the product peak in sodium chloride in an acetic acid buffer; wherein the radioactive concentration of the resulting solution is at least about 23 mCi/mL.

In another aspect, the invention features a product formed by the process for producing [¹²³I]-N-(3-iodoprop-2E-enyl)-2β-carbomethoxy-3β-(4-methylphenyl)nortropane.

In another aspect, the invention provides a sterile formulation of [¹²³I]-N-(3-iodoprop-2E-enyl)-2β-carbomethoxy-3β-(4-methylphenyl)nortropane produced by autoclaving at 121° C. (+2° C./−1° C.) for 20+/−1 minutes.

Imaging of the dopamine transporter enables the detection, assessment and monitoring of progression of dopamine-related disease and facilitates therapies directed to dopamine-related disease palliation and reversal. Exemplary therapies include implants of dopamine neurons and drugs that retard progression of the disease. A radiopharmaceutical that binds to the DAT provides clinical information to assist in the diagnosis and treatment of these various disease states.

### DETAILED DESCRIPTION OF THE INVENTION

For commercial production, it is useful to maximize radiolabel (e.g. 1231) incorporation into a final product as well as minimize the reaction time. It is also useful for safety of use that the final product has radiochemical and chemical purity acceptable to regulatory agencies. Furthermore, since initial reactants are less stable at low pH but the iodination reaction is optimal at low pH, care is taken to employ a process whereby the reaction period under acidic conditions is minimized.

Production is enhanced if the shelf life/stability can be lengthened. One method by which this can be accomplished is by increasing the final product's radioactive concentration. Since a radiolabel such as ¹²³I has a half-life of only 13.2 hours, extending the shelf life by an additional day suggests that the initial level of radioactivity should be increased about four-fold. Increased concentrations of radioactivity potentially reduce the stability of the product because of direct effects of radiation on the compound and by indirect effects caused by the generation of highly reactive compounds, including highly reactive compounds, from water. In one embodiment, a compound is one with the highest concentration of radioactive compound(s) that maintains sufficient chemical and radiochemical stability for the duration of use. It is to be understood that increasing radioactivity levels itself can increase compound degradation. Thus an elected final radioactivity level is a balancing test between stability and signal strength.

### DEFINITIONS

For convenience, certain terms employed in the specification, examples, and appended claims are collected here. Unless defined otherwise, all technical and scientific terms used herein have the same meaning as commonly understood by one of ordinary skill in the art to which this invention belongs. The initial definition provided for a group or term herein applies to that group or term throughout the present specification individually or as part of another group, unless otherwise indicated.

The articles “a” and “an” are used herein to refer to one or to more than one (i.e., to at least one) of the grammatical object of the article. By way of example, “an element” means one element or more than one element.

The term “or” is used herein to mean, and is used interchangeably with, the term “and/or,” unless context clearly indicates otherwise.

The term “carrier” is used herein to mean a non-radioactive version of a compound.

The term “radiochemical purity” is used herein to mean the fraction or percentage of the stated isotope present in the stated chemical form.

Radiochemical purity can be typically determined using an analytical chromatographic procedure (HPLC or ITLC) to separate the radioactive species in the formulation. Quantification of the various species using appropriate detection method allows calculation of radiochemical purity.

Radiochemical purity can be measured, for example, as a fraction or percentage of counts per unit time from desired product over total counts per unit time. See, Bioconjugate Chem, 2004, 15, 235-241 incorporated herein by reference. In a non-limiting example, a solution of PE2I having a radiochemical purity of at least 90%, represents a solution wherein no less than 90% of the radioactivity present in the product is tagged to PE2I. Thus, any other I-123 species may not be present at greater than 10% of the total radioactivity. In another non-limiting example, a solution containing the following radioactive materials-I-123 PE2I (90%), I-123 free iodide (5%) and I-123 labeled impurity present in the precursor (5%), the solution's radiochemical purity would be 90%.

The term “radiochemical yield” is the percentage of radioactive compound incorporated into a final product.

The term “substantially free of” is not meant to exclude trace amounts of a substance. Thus “substantially free of ethanol” means containing less than about 1% ethanol but not excluding trace amounts of ethanol. “Substantially free of ascorbic acid” means containing less than about 1% ascorbic acid but not excluding trace amounts of ascorbic acid. “Substantially carrier-free” means containing trace amounts of carrier but less than that which decreases binding of the radiochemical.

The terms “radiochemically stable,” “radiochemical stability,” or “stable,” is used herein to refer to the stability of the composition as a whole. As is recognized by those skilled in the art, use of the term stable or stability in the context of a radioactive compound is meant to indicate that chemical degradation of the molecule occurs to a limited extent and is not meant to suggest that radioactive decay processes are slowed or otherwise limited.

Tropane is a bicyclic tertiary amine compound C₈H₁₅N that is the parent compound of atropine, cocaine, and related alkaloids. Certain small organic molecules, some of which have high affinity and selectivity for the dopamine transporter (DAT), are useful in the diagnosis of Parkinson's disease (PD).

In one embodiment the tropane compound is as disclosed in U.S. Pat. No. 6,180,083. In one embodiment, the tropane compound is [¹²³I]-N-(3-iodoprop-2E-enyl)-2β-carbomethoxy-3β-(4-methylphenyl)nortropane. It is believed that, when given intravenously, [¹²³I]-N-(3-iodoprop-2E-enyl)-2β-carbomethoxy-3β-(4-methylphenyl) nortropane is able to penetrate the brain and bind to dopamine transport receptors.

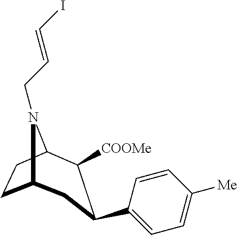

### [¹²³I] N-(3-iodoprop-2E-enyl)-2β-carbomethoxy-3β-(4-methylphenyl)-nortropane ([¹²³I] PE2I)

Other examples of imaging agents that target the dopamine transporter include [¹²³I]-2β-carbomethoxy-3β-(4 fluorophenyl)-N-(3-iodo-E-allyl)nortropane (Altropane®, Alseres Pharmaceuticals, Inc., Hopkinton, Mass.), [¹²³I]-N-ω-fluoropropyl-2β-carbomethoxy-3β-(4-iodophenyl)nortropane or Ioflupane (¹²³I) (DaTSCAN™, Nycomed-Amersham, Piscataway, N.J.), ¹¹C PE2I, (−)-2-β-Carbomethoxy-3-β-(4-fluorophenyl)tropane (β-CFT, WIN 35,428), (^{99m}Tc) 0-1505, and (^{99m}Tc)-Technepine. The above agents and other examples of useful DAT ligands include but are not limited to compounds disclosed in Fischman et al., 1998, Synapse, 29:125-41, Madras et al., 1996, Synapse 22:239-46; Meltzer et al., 1993, J. Med. Chem. 36:855-62; and Milius et al., 1990, J. Medicinal Chem. 34:1728-31, U.S. Pat. Nos. 5,493,026; 5,506,359; 5,770,180; 5,853,696; 5,948,933; 6,171,576; 6,548,041; 7,081,238; 6,180,083; 5,310,912; 5,439,666; 5,698,179; 5,750,089; 6,447,747; 6,537,522; 5,980,860; 6241963 and 6,180,083.

In one aspect, the invention features a diagnostic formulation comprising an aqueous solution comprising [¹²³I]-N-(3-iodoprop-2E-enyl)-2β-carbomethoxy-3β-(4-methylphenyl)nortropane, optionally wherein the aqueous solution is substantially carrier-free and substantially ascorbic acid-free.

In another embodiment, the aqueous solution is substantially radioprotectant-free.

In one embodiment, the aqueous solution comprises a radioactive concentration of at least about 15 and 18, and about 20 mCi/mL or more. In another embodiment, the aqueous solution comprises a radioactive concentration of at least about 23 mCi/mL.

In another embodiment, the aqueous solution comprises a radioactive concentration of at least about 1.6 mCi/mL at least about 50 hours post creation.

In one embodiment, the aqueous solution has a radiochemical purity of at least about 95%, and particularly at least about 97%.

In another embodiment, the aqueous solution comprises a concentration of ethanol in a percentage of less than about 10%, and less than about 5%, and further less than about 1%. In another embodiment, the aqueous solution is substantially ethanol-free.

In another embodiment, the aqueous solution comprises a pH of less than about 7. In another embodiment, the aqueous solution comprises a pH of less than about 6. In another embodiment, the aqueous solution comprises a pH ranging from about 2.5 to about 4.5.

In one embodiment, the [¹²³I]-N-(3-iodoprop-2E-enyl)-2β-carbomethoxy-3β-(4-methylphenyl)nortropane is radiochemically stable for at least 48 hours. In another embodiment, the [¹²³I]-N-(3-iodoprop-2E-enyl)-2β-carbomethoxy-3β-(4-methylphenyl)nortropane is radiochemically stable for at least about 60 hours.

In another aspect, the invention features a process for producing [¹²³I]-N-(3-iodoprop-2E-enyl)-2β-carbomethoxy-3β-(4-methylphenyl)nortropane. In one embodiment the process comprises the reaction of N-(3-tributyltin-2E-propenyl)-2β-carbomethoxy-3β-(4-methylphenyl)nortropane and sodium [¹²³I]-iodide. In another embodiment the process produces [¹²³I]-N-(3-iodoprop-2E-enyl)-2β-carbomethoxy-3β-(4-methylphenyl)nortropane in less than about 60 minutes, with greater than 95% radiochemical purity, a concentration of at least about 20 mCi/mL, a radiochemical yield of at least about 45% (and particularly at least about 65%, and at least about 75%.), without added carrier, and having a radiochemical and chemical stability sufficient for over about 50 hours, and particularly at least about 51 hours.

In another aspect, the invention features a process for producing an aqueous solution of [¹²³I]-N-(3-iodoprop-2E-enyl)-2β-carbomethoxy-3β-(4-methylphenyl) nortropane. In one embodiment, the solution is produced using a process comprising purification using hydrophobic media that allows separation and concentration. In another embodiment, the process comprises preparative HPLC purification. In one embodiment, the purification step is substantially free of a radiolysis inhibitor. In another embodiment, the purification step comprises the addition of a radiolysis inhibitor. In another embodiment, the purification step of the target compound is performed within 30 minutes.

Any suitable preparative HPLC system may be used but note is made of an HPLC column comprising packing material particles having an 18 carbon chain (C18). Examples of C18 columns include but are not limited to XTerra® C18 Column, (Waters Corp., Milford, Mass., See U.S. Pat. No. 6,686,035), and μBondpak C18 Column (Waters Corp., Milford, Mass.).

In one embodiment the process for producing [¹²³I]-N-(3-iodoprop-2E-enyl)-2β-carbomethoxy-3β-(4-methylphenyl)nortropane comprises the steps of:
a) Heating a basic solution (pH at least about 11) of sodium [¹²³I]-iodide to a range of about 70° C. to about 150° C.
b) Separately combining N-(3-tributyltin-2E-propenyl)-2β-carbomethoxy-3β-(4-methylphenyl)nortropane in great molar excess (about 0.05 to about 0.5 mg) in ethanol, an oxidizing agent (e.g., H₂O₂), and a buffer (e.g. sodium phosphate) at about pH 2.5 to 3.0
c) Acidifying the heated sodium [¹²³I]-iodide to a pH less than about 2 using an appropriate acid (e.g. trifluoroacetic acid) and adding the mixture defined in step (b)
d) Heating the mixture from (c) for about 20 minutes or less at a temperature ranging from about 70° C. to about 150° C.
e) Neutralizing the pH (e.g., by adding base such as NaOH) and treating the mixture with an oxidizing agent (e.g. sodium metabisulfite)
f) Purifying the [¹²³I]-N-(3-iodoprop-2E-enyl)-2β-carbomethoxy-3β-(4-methylphenyl)nortropane reaction product using hydrophobic media that allows separation and concentration (with or without radiolysis inhibitors) of the target compound within about 30 min, and
g) Diluting into an isotonic saline solution with acidic (less than about pH 7) buffer (e.g. phosphate) with or without radiolysis inhibitors (e.g. ascorbic acid) to a concentration of about 23 mCi/mL.
h) Sterilizing by autoclaving if the formulation buffer is less than about pH 6 (optionally pH about 2.5 to about 4.5). Optionally the solution at pH about 2.5 to about 7.0 may be sterilized by filtration (note: any lower limitation on useful pH is a function of degree of injection discomfort and not due to chemical instability).

In another aspect, the invention features a product formed by the process for producing [¹²³I]-N-(3-iodoprop-2E-enyl)-2β-carbomethoxy-3β-(4-methylphenyl)nortropane. In one embodiment the product formed by the process of preparing a precursor solution N-(3-tributyltin-2E-propenyl)-2β-carbomethoxy-3β-(4-methylphenyl)nortropane, ethanol, hydrogen peroxide, and phosphate buffer; preparing a sodium [¹²³I]-iodide solution comprising sodium [¹²³I]-iodide and trifluoroacetic acid having a pH of less than about 2; heating a mixture of precursor solution and sodium [¹²³I]-iodide solution at a temperature of about 80° C. for about 15 minutes; eluting the [¹²³I]-N-(3-iodoprop-2E-enyl)-2β-carbomethoxy-3β-(4-methylphenyl)nortropane through a C18 preparative HPLC column with an eluent, wherein the eluent comprises about 15% (v/v) ethanol; and collecting the product peak in sodium chloride in an acetic acid buffer.

### EXAMPLES

### Example 1

### Synthesis of [¹²³I]-N-(3-iodoprop-2E-enyl)-2β-carbomethoxy-3β-(4-methylphenyl)nortropane

Sodium [¹²³I]-iodide (4 Ci) in 0.1N NaOH is dispensed in a 10 mL vial and heated to about 80° C. Phosphate buffer, 0.80 mL 0.1 M, pH 2.5-3.0, is combined with 0.20 mL 30% hydrogen peroxide, and 0.50 mL of 1 mg/mL (in ethanol) N-(3-tributyltin-2E-propenyl)-2β-carbomethoxy-3β-(4-methylphenyl)nortropane to form a precursor containing mixture. The sodium [¹²³I]-iodide solution is acidified (final pH<2) by the addition of trifluoroacetic acid. The precursor-containing mixture is added to the acidified sodium [¹²³I]-iodide solution. The mixture is heated at 80° C. for 15 minutes.

After 15 minutes, 2 mL of sodium metabisulfite solution is added to stop the reaction (100 mg/mL in Sterile Water for Injection). One mL of a 100 mg/mL solution of Ascorbic Acid is added to the reaction mixture as a radioprotectant. The acidic reaction mixture is optionally neutralized with 500 μL of 5 N Sodium Hydroxide. After neutralization, the pH is >6. Neutralization may be optional if the subsequent HPLC system is not degraded too quickly by the low pH and oxidant.

### Example 2

### Chromatography of [¹²³I]-N-(3-iodoprop-2E-enyl)-2β-carbomethoxy-3β-(4-methylphenyl)nortropane

The reaction mixture of Example 1 is transferred to a preparative HPLC system (XTerra® C18 Column from Waters Corp., Milford, Mass., see U.S. Pat. No. 6,686,035).

### XTerra® Column

### Packing Material: C-18

### Particle Size: 5 μm

### Length: 50 mm

### Diameter: 10 mm

### Column Volume: 4 mL

[¹²³I]-N-(3-iodoprop-2E-enyl)-2β-carbomethoxy-3β-(4-methylphenyl)nortropane is eluted using the following eluent system: isocratic elution buffer, 15% (v/v) ethanol, 85% 10 mM glacial acetic acid in sterile water for injection. The product peak is collected into a vessel containing sodium chloride injection (USP) in an acetic acid buffer pH 2.5 to 3.5, and, due to carry over, the final solution has about 1.8% ethanol. Noted is the fact that in particular embodiments, the product comprises an aqueous solution comprising 0.1-5 mCi/mL, at the time of production, [¹²³I]N-(3-iodoprop-2E-enyl)-2β-carbomethoxy-3β-(4-methylphenyl)nortropane, 3.5-10% ethanol, 0.2-0.4 mg/mL ascorbic acid, 5-50 μM glacial acetic acid, sodium hydroxide buffer, pH 2.5-3.5, and 0.85-0.95% sodium chloride. Also noted are embodiments wherein the product comprises an aqueous solution comprising 0.1-23 mCi/mL, at the time of production, [¹²³I]-N-(3-iodoprop-2E-enyl)-2β-carbomethoxy-3β-(4-methylphenyl)nortropane, 1.5-10% ethanol, 0-0.4 mg/mL ascorbic acid, 5-50 μM glacial acetic acid, sodium hydroxide buffer, pH 2.5-3.5, and 0.85-0.95% sodium chloride.

The instant resulting radioactive concentration of [¹²³I]-N-(3-iodoprop-2E-enyl)-2β-carbomethoxy-3β-(4-methylphenyl)nortropane is about 23 mCi/mL.

### Example 3

### Chromatography of [¹²³I]-N-(3-iodoprop-2E-enyl)-2β-carbomethoxy-3β-(4-methylphenyl)nortropane

The reaction mixture of Example 1 is transferred to a preparative HPLC system (μBondpak® C18 Column from Waters Corp., Milford, Mass.).

### μBondpak Column

### Packing Material: C-18

### Particle Size: 10 μm

### Length: 300 mm

### Diameter: 19 mm

### Column Volume: 85 mL

[¹²³I]-N-(3-iodoprop-2E-enyl)-2β-carbomethoxy-3β-(4-methylphenyl)nortropane is eluted using the following eluent system: isocratic elution buffer, 80% (v/v) ethanol, 20% ascorbic acid in sterile water for injection 20 g/L. The product peak is collected into a vessel containing sodium chloride injection (USP) in an acetic acid buffer pH 2.5 to 3.5. Due to carry over the final solution has about 3.8 to about 6.3% ethanol and about 0.2 to about 0.4 g/L ascorbic acid. The resulting radioactive concentration of [¹²³I]-N-(3-iodoprop-2E-enyl)-2β-carbomethoxy-3β-(4-methylphenyl)nortropane is about 5 mCi/mL of solution.

### Example 4

### Dilution of [¹²³I]-N-(3-iodoprop-2E-enyl)-2β-carbomethoxy-3β-(4-methylphenyl)nortropane

The chromatographed mixture of Example 2 is adjusted by dilution with acetic acid buffer, pH 2.5 to 4.5, to produce an aqueous solution comprising 16 mCi/mL, (at the time of production), [¹²³I]N-(3-iodoprop-2E-enyl)-2β-carbomethoxy-3β-(4-methylphenyl)nortropane, 4% ethanol, 10 μM glacial acetic acid, sodium hydroxide buffer, pH 2.5-3.5, and 0.9% sodium chloride.

### Example 5

### Dilution of [¹²³I]-N-(3-iodoprop-2E-enyl)-2β-carbomethoxy-3β-(4-methylphenyl)nortropane

The chromatographed mixture of Example 3 is adjusted by dilution with acetic acid buffer, pH 2.5 to 4.5, to produce an aqueous solution comprising 4 mCi/mL (at the time of production), [¹²³I]-N-(3-iodoprop-2E-enyl)-2β-carbomethoxy-3β-(4-methylphenyl)nortropane, 4% ethanol, 0.3 mg/mL ascorbic acid, 10 μM glacial acetic acid, sodium hydroxide buffer, pH 2.5-3.5, and 0.9% sodium chloride.

### Example 6

### Sterilization of [¹²³I]-N-(3-iodoprop-2E-enyl)-2β-carbomethoxy-3β-(4-methylphenyl)nortropane

The solution of Example 3 is aliquoted into 10 mL glass vials which are then placed in an autoclave and autoclaved at 121° C. (+2° C./−1° C.) for 20+/−1 minutes. Acceptable radiochemical purity of the product is shown by HPLC and sterility testing confirms the sterility of the resultant product.

### Example 7

### Parkinson's Diagnosis

A 64 year old conventionally diagnosed with PS is dosed with 0.14 μg of tracer/100 g of body weight of [¹²³I]-N-(3-iodoprop-2E-enyl)-2β-carbomethoxy-3β-(4-methylphenyl)nortropane at 1 mCi activity.

A SPECT scan is performed. Strong evidence of different nonspecific binding in the striatal region, compared with the reference region is found, leading to bias in the estimate of dopaminergic denervation (range, 36%-94% severity) was found. One percent transporter occupancy was reached with 0.14 μg of tracer/100 g of body weight, corresponding to an SA of 5.7 kBq/pmol for the given radioactivity dose, and 10% occupancy was reached at 1.5 μg of tracer/100 g of body weight, corresponding to an SA of 0.57 kBq/pmol. The 6-hydroxydopamine lesion affected B(max) (control, 402±94 pmol/mL; lesioned, 117±120 pmol/mL; P=0.003) but not K app d (control, 331±63 pmol/mL; lesioned, 362±119 pmol/mL; P=0.63).

### Example 8

### Lewy Body Dementia Diagnosis

SPECT imaging of the dopamine transporter with ¹²³I-PE2I is conducted on six subjects previously diagnosed with Lewy Body Dementia, and on control individuals without a diagnosis of Lewy Body Dementia. For each individual tested, greater than 1 mCi of ¹²³I-PE2I is administered by intravenous injection at the onset of imaging. Images of the striatum are collected and analyzed by a radiologist to determine striatal binding potentials. In general, the methodology used for the SPECT imaging is the same as the methods described in Fischman et al., 1998, Synapse 29:125 41, which is incorporated herein by reference. As determined by the imaging, these six Lewy Body Dementia individuals show reduced binding potential and, therefore, reduced dopamine transporter levels compared to expected levels for aged matched normal individuals.

### Example 9

### ADHD Diagnosis

SPECT imaging of the dopamine transporter with 1231-PE2I is conducted on six subjects previously diagnosed with ADHD, and on control individuals without a diagnosis of ADHD. For each individual tested, greater than 1 mCi of 1231-PE2I is administered by intravenous injection at the onset of imaging. Images of the striatum are collected and analyzed by a radiologist to determine striatal binding potentials. In general, the methodology used for the SPECT imaging is the same as the methods described in Fischman et al., 1998, Synapse 29:125-41, which is incorporated herein by reference. As determined by the imaging, these six ADHD individuals show reduced binding potential and, therefore, reduced dopamine transporter levels compared to expected levels for aged matched normal individuals.

All publications, patent applications, patents, and other references mentioned herein are incorporated by reference in their entirety. In addition, the materials, methods, and examples are illustrative only and not intended to be limiting. Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below.

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A formulation comprising an aqueous solution comprising [¹²³I]-N-(3-iodoprop-2E-enyl)-2β-carbomethoxy-3β-(4-methylphenyl)nortropane, wherein the solution comprises a radioactive concentration of at least about 18 mCi/mL.

2. The formulation of claim 1, wherein the radioactive concentration is at least about 23 mCi/mL.

3. A formulation comprising an aqueous solution comprising [¹²³I]-N-(3-iodoprop-2E-enyl)-2β-carbomethoxy-3β-(4-methylphenyl)nortropane, wherein the solution comprises a radioactive concentration of at about 4 mCi/mL.

4. A formulation comprising an aqueous solution comprising [¹²³I]-N-(3-iodoprop-2E-enyl)-2β-carbomethoxy-3β-(4-methylphenyl)nortropane, wherein the solution comprises a radioactive concentration of at about 16 mCi/mL.

5. The formulation of claim 1, wherein the formulation exhibits radioactive concentration of at least about 1.6 mCi/mL at least about 51 hours post creation.

6. The formulation of claim 1, wherein the aqueous solution comprises a pH of less than about 7.

7. The formulation of claim 1, wherein the aqueous solution comprises a pH of less than about 6.

8. The formulation of claim 1, wherein the aqueous solution comprises a pH ranging from about 2.5 to about 4.5.

9. The formulation of claim 1, wherein the aqueous solution comprises a radiochemical purity of at least about 95%.

10. The formulation of claim 1, wherein the aqueous solution comprises a concentration of ethanol of less than about 10%.

11. The formulation of claim 1, wherein the aqueous solution comprises a concentration of ethanol of less than about 5%.

12. The formulation of claim 1, wherein the aqueous solution comprises a concentration of ethanol of less than about 1%.

13. The formulation of claim 1, wherein the aqueous solution is substantially free of ethanol.

14. The formulation of claim 1, wherein the aqueous solution is substantially carrier free.

15. The formulation of claim 1, wherein the aqueous solution is substantially ascorbic acid free.

16. A method of preparing [¹²³I]-N-(3-iodoprop-2E-enyl)-2β-carbomethoxy-3β-(4-methylphenyl)nortropane comprising the steps of:
a. Preparing a precursor solution comprising N-(3-tributyltin-2E-propenyl)-2β-carbomethoxy-3β-(4-methylphenyl)nortropane, ethanol, hydrogen peroxide, and phosphate buffer;
b. Preparing a sodium [¹²³I]-iodide solution comprising sodium [¹²³I]-iodide and trifluoroacetic acid having a pH of less than about 2; and
c. Heating a mixture of precursor solution and sodium [¹²³I]-iodide solution at a temperature of about 80° C. for about 15 minutes.

17. A method of preparing an aqueous solution of [¹²³I]-N-(3-iodoprop-2E-enyl)-2β-carbomethoxy-3β-(4-methylphenyl)nortropane comprising the steps of:
a. eluting the [¹²³I]-N-(3-iodoprop-2E-enyl)-2β-carbomethoxy-3β-(4-methylphenyl)nortropane through a C 18 preparative HPLC column with an eluent, wherein the eluent comprises about 15% (v/v) ethanol; and
b. Collecting the product peak in sodium chloride in an acetic acid buffer, wherein the radioactive concentration of the resulting solution is at least about 23 mCi/mL.

18. A method of preparing an aqueous solution of [¹²³I]-N-(3-iodoprop-2E-enyl)-2β-carbomethoxy-3β-(4-methylphenyl)nortropane comprising the steps of:
a. Eluting a solution of [¹²³I]-N-(3-iodoprop-2E-enyl)-2β-carbomethoxy-3β-(4-methylphenyl)nortropane through a C18 preparative HPLC column with an eluent, wherein the eluent comprises about 80% (v/v) ethanol, and about 20% ascorbic acid (20 g/L) in sterile water for injection; and
b. Collecting the product peak in sodium chloride in an acetic acid buffer, wherein the radioactive concentration of the resulting solution is at least about 20 mCi/mL.

19. The product of the process comprising the steps of:
a. Preparing a precursor solution comprising N-(3-tributyltin-2E-propenyl)-2β-carbomethoxy-3β-(4-methylphenyl)nortropane, ethanol, hydrogen peroxide, and phosphate buffer;
b. Preparing a sodium [¹²³I]-iodide solution comprising sodium [¹²³I]-iodide and trifluoroacetic acid having a pH of less than about 2;
c. Heating a mixture of precursor solution and sodium [¹²³I]-iodide solution at a temperature of about 80° C. for about 15 minutes;
d. Eluting the [¹²³I]-N-(3-iodoprop-2E-enyl)-2β-carbomethoxy-3β-(4-methylphenyl)nortropane through a C 18 preparative HPLC column with an eluent, wherein the eluent comprises about 15% (v/v) ethanol; and
e. Collecting the product peak in sodium chloride in an acetic acid buffer.

20. A sterile formulation of [¹²³I]-N-(3-iodoprop-2E-enyl)-2β-carbomethoxy-3β-(4-methylphenyl)nortropane produced by autoclaving.

21. The sterile formulation of claim 20 wherein the autoclaving is performed for about 19 to 21 minutes at a temperature of about 120° C. to 123° C.

22. The sterile formulation of claim 20 wherein the autoclaving is performed for about 20 minutes at a temperature of about 121° C.

23. A process for producing a sterile formulation of [¹²³I]-N-(3-iodoprop-2E-enyl)-2β-carbomethoxy-3β-(4-methylphenyl)nortropane comprising autoclaving a solution of [¹²³I]-N-(3-iodoprop-2E-enyl)-2β-carbomethoxy-3β-(4-methylphenyl)nortropane.
